(19) United States
(12) Patent Application Publication
(10) Pub. No.: US 2023/0124338 A1
(43) Pub. Date: Apr. 20, 2023

(54) ASPJ Pull-On's

(71) Applicant: Brenda H. Pate, Cypress, TX (US)

(72) Inventor: Brenda H. Pate, Cypress, TX (US)

(21) Appl. No.: 17/505,850

(22) Filed: Oct. 20, 2021

(51) Int. Cl. A61F 13/42; A61F 13/496; A61B 10/00

(52) U.S. Cl. CPC A61F 13/42; A61F 13/496; A61B 10/0038; A61F 2013/15154

(57) ABSTRACT

The present invention is a disposable brief made of absorbent material with small amount of cellulose for good absorbance. The material has elasticity for comfort on the skin and stretch ability. The said invention was designed for full coverage to prevent any leakage from the anterior and posterior of the perinium. Velcro fastening on the lateral sides for easy removal and applying if needed. The said invention has a pocket in the front and back of the disposable material brief for the insertion of FDA approved Urine test strips for the anterior and FDA approved Guaiac paper that can be inserted in the posterior part of the brief. The brief was designed to be a funnel for testing material for early detection of urinary tract infections and gastrointestinal bleeding. Urine test strips and Guaiac paper purchased separately.

### 1. BACKGROUND OF THE INVENTION

As a Registered Nurse I have saw so many lives lost due to untreated UTI Infections that lead to Sepsis then Sepsis causes organ failure or death. I have also witnessed multiple falls and injury and death due to low blood count missed while people where at home or in nursing homes and was unable to express themselves due to cognitive disfunction or just lack of medical knowledge. ASPJ Pull-on will initiate early detection. All those who are bedbound, incontinent and nonverbal will benefit for this said invention. It's a smart approach to stop one of the leading causes of death. Once incontinence occurs and goes onto the urine test strip it gives and indicator for the patient or caregiver to call their primary care physician so a qualified person can diagnose. When the stool gets on the guaiac paper the patient or caregiver can put it in a zip lock bag and take it to their physician for further testing. This is a proactive approach to infection to incontinent individuals.

### 1. DESCRIPTION OF DRAWING

FIG. 1 shows the front part of the brief. It shows a pocket designed inside the brief for the insertion of PH paper or ect. Littman urine strip that are sold separately that detects a suspicion of an infection. The pocket for the actual design is the size of 2 in in length and the opening is stretchable to 2 in W for the average fingers to remove the test strip to read.

FIG. 2 shows the back of the brief that also has a pocket that will allow testing inserts. The size 3×2 long enough for the average stool testing paper to fit.

FIG. 3 shows the inside of the Pull on delivering the vision of a diaper, no genitals will be irritated with testing material

FIG. 4 shows the pocket and the opening, delivering a visual that it is room enough to insert testing material

FIG. 5 shows the thickness of the absorbable material.

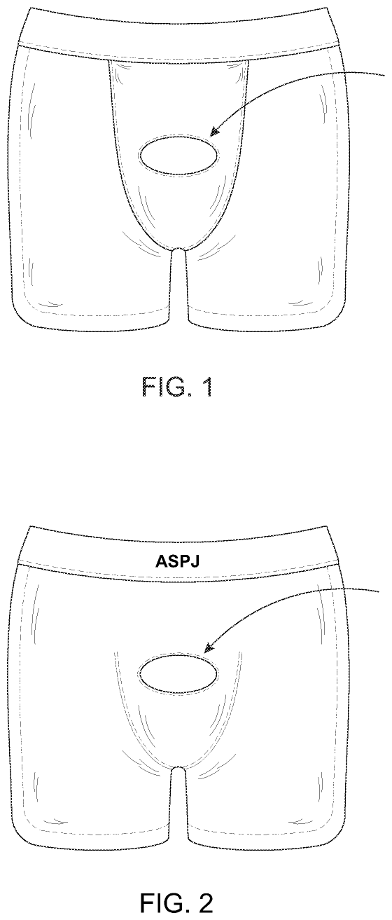

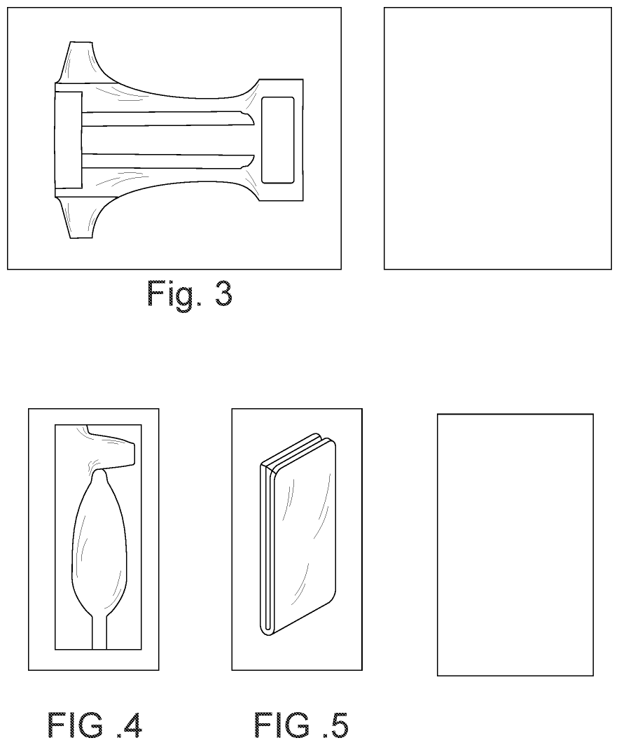

### 1. DETAILED DESCRIPTION OF THE INVENTION

The invention is created with stretchable absorbable material. It provides full coverage in the shape of a brief resembling fitted shorts they start from below the umbilicus to mid-thigh. The lateral sides come with Velcro fastening for applying and removing. They also come as a pull on for those who are ambulatory. The material is white, the front part of the brief as a pocket that is 2×2 in. the back has a pocket that is 3×2. The material has small amounts of cellulose for absorption. The perinium area is 1 in thick and the lateral sides is less than ¼ thickness for comfort and thin enough for wearing under clothing. The invention weighs 1.4-25 oz according to sizes. The present invention relates to absorbent products such as diapers, training pants or any incontinent garments. Traditional products are folded in the front, this present invents fasten on the lateral sides.

1-8. (canceled)

9. A Pull-On diaper apparatus comprising of a polyester, cellulose, nonwoven fabric with elastic and Velcro materials that soaks up to 300 ml of urine;
Sizes ranges from infants to adults according to weight in pounds: the said Pull-On is easily removed on bed bound individuals.

10. The invention of claim 9 further comprising of a pocket in the frontal portion of the said Pull-On that allows for FDA approved testing paper to alert the individual wearing the said Pull-On of possible infection and to contact their provider.

11. The invention of claim 10 further comprising of a pocket in the posterior portion of the said Pull-On to collect stool to be taken to a physician for further testing of gastrointestinal bleeding.

12. The invention of claim 10 wherein the front pocket comprising of PH testing paper changes color according to the acidity of the urine; changes in color cause the alert for a physician to be notified to prevent sepsis for occurring.

13. The invention of claim 11 wherein the posterior pocket designed for the FDA approved Guaiac paper for stool collection when individual have a bowel movement; stool is to be delivered to their physician for further testing of gastrointestinal bleeding.
